(19) United States
(12) Patent Application Publication
(10) Pub. No.: US 2003/0096823 A1
(43) Pub. Date: May 22, 2003

(54) Method for the treatment of cardiotoxicity induced by antitumor compounds

(76) Inventors: Beryl Asp, Bridgewater, NJ (US); Angela Ongden, Long Valley, NJ (US); Langdon Miller, Lebanon, NJ (US); Arturo Podesta, Milano (IT)

Correspondence Address: Lisa M.W. Hillman, McDonnell Boehnen Hulbert & Berghoff, 32nd Floor, 300 S. Wacker Drive, Chicago, IL, 60606, US

(21) Appl. No.: 09/993,211

(22) Filed: Nov. 16, 2001

(51) Int. Cl. A61K 31/496; A61K 39/395

(52) U.S. Cl. 514/252.11; 424/155.1

(57) ABSTRACT

The present invention relates to a method for treating a cancer, which comprises administering a therapeutically effective amount of trastuzumab alone or in association with an anthracycline to a patient in need thereof, in combination with an amount of dexrazoxane effective to ameliorate cardiotoxicity.

### SUMMARY

The present invention relates to a method for treating a cancer, especially a cancer overexpressing human epidermal growth factor receptor 2 (HER2), which comprises administering a therapeutically effective amount of trastuzumab alone or in association with an anthracycline, in combination with an amount of dexrazoxane effective to ameliorate cardiotoxicity. A method for ameliorating cardiotoxic effects caused by trastuzumab when administered alone or in combination with an anthracycline is also within the scope of the invention.

### BACKGROUND

Trastuzumab is a recombinant DNA-derived humanized monoclonal antibody that selectively binds with a high affinity in cell-based assays (Kd: 5 nM) to the extracellular domain of the human epidermal growth factor receptor 2 protein, HER2. The antibody is an IgG₁ kappa that contains human framework regions with the complementary-determining regions of a murine antibody (4D5) that binds to HER2.

HER2 (or c-erbB2) proto-oncogene encodes a transmembrane receptor protein of 185 kDa, which is structurally related to the epidermal growth factor receptor.

Trastuzumab has been shown, in both in vitro assays and animal models, to inhibit the proliferation of human tumor cells from different sources overexpressing HER2. Furthermore, nonclinical studies have shown additive and synergistic effects of trastuzumab when given in combination with several chemotherapeutic agents including anthracyclines.

HER2 protein overexpression is observed in patients with breast cancers and other malignancies, including those arising from the uterine endometrium, pancreas, colon, ovaries, lung, stomach, salivary glands, and head and neck tumors. In patients with breast cancers, HER2 overexpression seems to correlate with a poor prognosis because of the high growth rates of tumors.

In large, multicenter trials of trastuzumab as a single agent or in combination with chemotherapy as first-line or second-line therapy for metastatic breast cancer (MBC), response rates have ranged from 12% to 23% for single-agent trastuzumab and from 25% to 62% for trastuzumab plus chemotherapy.

Trastuzumab alone is generally a well tolerated drug. However, an overview of clinical data indicates that its use is sometimes associated with an undesired cardiotoxicity, clinically expressed, as for anthracyclines, by a progressive decrease in cardiac systolic function or even by a serious damage of myocytes, mainly in the left ventricle and septum.

Anthracyclines, a well known class of compounds in the antineoplastic group of agents include, e.g., doxorubicin, daunorubicin, epirubicin (4′epi-doxorubicin) and idarubicin (4′ demethoxy-daunorubicin).

The histomorphology of anthracycline-induced cardiotoxicity has been well characterized in several animal species and closely resembles that in humans. The cardiomyopathy is characterized by multifocal vacuolar degeneration of myocytes. Dilation of sarcoplasmic reticulum and transverse tubules has also been described. As for trastuzumab, the myocardial damage is generally more evident in the left ventricle and septum. The mechanism underlying anthracycline-induced cardiotoxicity has not been conclusively determined, but considerable evidence has accumulated indicating that cardiomyopathy is principally due to an iron-dependent free radical oxidative stress.

The risk of cardiotoxicity associated with anthracyclines represents a limitation to the optimal use of this class of chemotherapeutic agents in humans. Consequently, much research has been directed at the identification and characterization of potential antidotes that prevent or reduce the development of cardiomyopathy. Among them, dexrazoxane (also known as ICRF-187), a bis-diketopiperazine derivative, structurally related to ethylenediamine tetracetic acid, has been shown to be effective in ameliorating the cardiotoxicity induced in experimental animals and humans by anthracycline compounds. Although the mechanism by which dexrazoxane reduces anthracycline-induced cardiotoxicity has not been fully elucidated, it would appear that the compound, unlike other free radical scavengers, specifically disrupts the drug-iron complexes that can bind to DNA and membrane targets: for which the latter acts as a source for hydroxyl radicals. In addition, dexrazoxane can be expected to also effectively chelate adventitious iron.

Speyer's U.S. Pat. Nos. 5,242,901 and 5,744,455 disclose a method of preventing an anthracycline-induced cardiotoxicity by using dexrazoxane and a method of treating cancer by administration of dexrazoxane and an anthracycline.

Creighton's U.S. Pat. No. 4,275,063 discloses anticancer pharmaceutical compositions for aiding regression and palliation of sarcoma, lymphosarcoma and leukaemia in humans. Therapeutically effective amounts of the compositionsin aid in regression and palliation of dexrazoxane.

An increased incidence and severity of cardiotoxicity was observed in patients receiving either concomitanly or sequentially trastuzumab with an anthracycline.

Even though several studies have been undertaken to learn the nature of trastuzumab-induced cardiotoxicity, its mechanism of action, as well as the mechanism by which trastuzumab potentiates the cardiotoxic effects of anthracyclines, are still unclear and are not fully elucidated.

It is highly desirable to exploit the full potential of trastuzumab alone or in combination of an anthracycline, by reducing the risk of cardiotoxicity without substantially affecting the anti-tumor activity of these drugs.

There is therefore a need in the art for a therapy for amelioratig cardiotoxicity induced by transtuzumab administered alone or in combination with an anthracycline.

It has now been found that dexrazoxane can have cardioprotective efficacy not only on trastuzumab-induced myocardial damage, but also on myocardial damage caused by its concomitant or sequential administration with an anthracycline.

### DETAILED DESCRIPTION OF THE INVENTION

It is an object of the present invention to provide a method for treating a cancer, which comprises administering a therapeutically effective amount of trastuzumab to a patient in need thereof, in combination with an amount of dexrazoxane effective to ameliorate cardiotoxicity.

The invention further comprises a method for treating a cancer, which comprises administering a therapeutically effective amount of trastuzumab in association with an anthracycline to a patient in need thereof, in combination with an amount of dexrazoxane effective to ameliorate cardiotoxicity.

The term “cancer” as used herein, unless otherwise indicated, means a cancer overexpressing the human epidermal growth factor receptor 2 (HER2), such as, for example, breast cancers and other malignancies, including those arising from the uterine endometrium, pancreas, colon, ovaries, lung, stomach, salivary glands, and head and neck tumors. In patients with breast cancers, HER2 overexpression seems to correlate with a poor prognosis because of the high growth rates of tumors.

The term “anthracycline” as used herein, unless otherwise indicated, means doxorubicin, daunorubicin, epirubicin (4′epi-doxorubicin) and idarubicin (4-demethoxy-daunorubicin). A particularly preferred anthracycline according to the invention is epirubicin.

A further object of the present invention provides a method for treating a cancer, which comprises administering a therapeutically effective amount of trastuzumab in association with epirubicin, in combination with an amount of dexrazoxane effective to ameliorate cardiotoxicity.

A method for ameliorating cardiotoxic effects caused by trastuzumab when administered alone is also within the scope of the invention.

In a further aspect, the present invention is directed to a method for ameliorating cardiotoxic effects caused by trastuzumab when administered in combination with an anthracycline, especially epirubicin.

In another aspect, the invention relates to the use of dexrazoxane in the manufacture of a medicament for use in ameliorating the cardiotoxicity induced by trastuzumab.

In a still another aspect, the invention relates to the use of dexrazoxane in the manufacture of a medicament for use in ameliorating the cardiotoxicity induced by trastuzumab in conjunction with an anthracycline, especially epirubicin.

The effectiveness of the treatment can be determined experimentally by controlled pre-clinical trials to assess the cardiotoxic potential of a recombinant murine anti-HER2 antibody (rmuAb) when given alone or in combination with a representative compound of the anthracycline class, e.g. epirubicin. A validated mouse model (e.g., Bertazzoli mouse model) can be used in pre-clinical trials. Pre-clinical trials can also be used to verify amelioration of cardiotoxicity by dexrazoxane of the single and combined effects on the heart by the recombinant antibodies and anthracyclines. The use of a murine HER2 antibody instead of trastuzumab is needed because trastzumab is specific for human and primate HER2. The efficacy of the combination therapy in alleviating the signs and symptoms of cardiotoxicity will be compared with the therapy without dexrazoxane.

The following protocol illustrates but does not limit the scope of the invention.

Protocol Scheme

Study title: Effect of Dexrazoxane on the Cardiotoxicity Induced by a Recombinant Murine Anti-HER2 Antibody Alone or in Combination with Epirubicin in the Bertazzoli Mouse Model.

Background and purpose: Trastuzumab is a recombinant humanized anti-HER2 antibody in clinical use for the treatment of breast cancers overexpressing HER2. In humans, it has been reported to produce cardiac toxicity per se and to worsen the myocardial damage produced by doxorubicin and epirubicin (epi). Dexrazoxane (dex) is a bis-diketopiperazine derivative, structurally related to ethylenediamine tetracetic acid (EDTA) which, among other activities, has been shown to be effective in ameliorating the cardiotoxicity induced by the above anthracyclines. The aim of this study is to confirm the cardiotoxic effect of a recombinant murine anti-HER2 antibody (rmuAb) when given alone or in combination with epi in a validated mouse model and to verify if dex is able to ameliorate their single and combined toxic effects on the heart.

Animals/group: 15 Crl:CD-1 (ICR) BR female mice, about 4 weeks old at the start of treatment, will be used.

Test and control articles and formulations: Epi and rmuAb will be dissolved in normal saline (saline) and distilled water for injection (water), respectively, at the requested concentrations; dex will be dissolved in M/6 sodium lactate at the requested concentration; control animals will receive the above vehicles alone.

Dose levels and justification: The doses of 5 mg/kg/day for epi and 50 mg/kg/day for dex (epi/dex ratio: 1:10) are selected on the basis of the results of previous cardiotoxicity studies in mice. 5 mg/kg/day epi is expected to induce a moderate/marked cardiotoxicity and 50 mg/kg/day dex is expected to significantly reduce the myocardial damage produced by epi. The two doses of rmuAb will be decided on the basis of the results of two preliminary studies performed to assess its toxicity and cardiotoxic potential in the above strain of mice.

Administration route: intravenous, via a tail vein, for epi, dex and rmuAb.

Study duration and treatment schedule: Twice a week at weeks 1, 2, 5, 6, and 7 for epi and dex; twice a week for 7 weeks for rmuAb. Dex will be given 30 minutes before epi. The interval of administration between epi/dex and rmuAb at weeks 1, 2, 5, 6, and 7 will be decided on the basis of the results of study No. 2 in the same mouse model. Probably, rmuAb will be given after epi. In the negative and positive control groups (groups 1-4), lactate, saline and water will be administered according to the treatment schedules adopted for the respective compounds. Surviving animals will be killed at the end of a 4-week observation period.

Experimental Groups:

1. lactate+saline+water

2. lactate+epi+water

3. lactate+saline+rmuAb low dose

4. lactate+saline+rmuAb high dose

5. lactate+epi+rmuAb low dose

6. lactate+epi+rmuAb high dose

7. dex+saline+rmuAb low dose

8. dex+saline+rmuAb high dose

9. dex+epi+rmuAb low dose

10. dex+epi+rmuAb high dose

Clinical observations and post-mortem examinations: Mortality, clinical signs and general condition will be recorded daily and body weight weekly. Decedent and killed animals will be autopsied. The heart of each animal will be removed, immediately placed in paraformaldehyde, and weighed. After fixation, the hearts will be embedded in plastic, sectioned and stained for histological examination. Cardiomyopathy will be evaluated by scoring the severity and extent of the myocardial lesions according to a qualitative/quantitative score. Heart mean total scores (MTS) and heart weights will be statistically compared.

The results obtained by the above study can be useful to demonstrate the effectiveness of dexrazoxane in ameliorating cardiotoxicity induced by trastuzumab alone or in combination with a drug belonging to anthracycline class, especially epirubicin.

In the above methods, the therapeutically effective amount of trastuzumab in a patient can be from about 0.1 mg/kg to about 100 mg/kg, preferably from about 1 mg/kg to about 10 mg/kg; the anthracycline can be from about 0.1 mg/m2 to about 1000 mg/m2, preferably from about 0.5 mg/m2 to about 500 mg/m2; dexrazoxane can be a dexrazoxane: anthracycline ratio from about 1:1 to about 100:1, preferably from about 5:1 to about 30:1.

However, it will be understood that the therapeutic dosage administered will be determined by the physician in the light of the relevant circumstances including the severity of the condition to be treated and the chosen route of administration. Therefore, the above dosage ranges are not intended to limit the scope of the invention in any way. Dexrazoxane can be administered, separately, sequentially or simultaneously with trastuzumab or with a selected anthracycline in any desired order.

A combined preparation for simultaneous, separate, or sequential administration for ameliorating cardiotoxic effects caused by the administration of trastuzumab alone or in combination with an anthracycline is also within the scope of the invention.

Since the present invention relates to a treatment with a combination of two or more active ingredients, which can be administered concomitantly or separately, the invention also relates to combining separate pharmaceutical compositions in a kit form. The kit includes two or three separate pharmaceutical compositions: trastuzumab and/or an anthracycline and dexrazoxane. The kit includes a container for containing the separate compositions such as a divided bottle or a divided foil packet, however, the separate compositions can also be contained within a single, undivided container.

A kit comprising:

a. trastuzumab and a pharmaceutically acceptable carrier or diluent in a first unit dosage form;

b. dexrazoxane and a pharmaceutically acceptable carrier or diluent in a second unit dosage form; and

c. a container, is also within the scope of the invention.

A kit comprising:

a. trastuzumab and a pharmaceutically acceptable carrier or diluent in a first unit dosage form;

b. an anthacycline and a pharmaceutically acceptable carrier or diluent in a second unit dosage form;

c. dexrazoxane and a pharmaceutically acceptable carrier or diluent in a third unit dosage form; and

d. a container, is also within the scope of the invention.

In particular, a kit comprising:

a. trastuzumab and a pharmaceutically acceptable carrier or diluent in a first unit dosage form;

b. epirubicin and a pharmaceutically acceptable carrier or diluent in a second unit dosage form;

c. dexrazoxane and a pharmaceutically acceptable carrier or diluent in a third unit dosage form; and

d. a container, is also within the scope of the invention.

Pharmaceutical compositions according to the invention can be prepared, for example, as parenteral, oral or transdermal dosage forms.

Compositions of the invention containing a pharmaceutically acceptable carrier can be prepared by any of the well known techniques of pharmacy that comprise admixing the excipients with a drug or therapeutic agent.

The term “ameliorating” as used herein, unless otherwise indicated, means reversing, alleviating, inhibiting the progress of, or preventing the disorder or condition to which such term applies, or one or more symptoms of such disorder or condition. In a preferred aspect, the term “ameliorating” means preventing.

1. A method for treating a cancer, which comprises administering a therapeutically effective amount of trastuzumab to a patient in need thereof, in combination with an amount of dexrazoxane effective to ameliorate cardiotoxicity.

2. A method for treating a cancer, which comprises administering a therapeutically effective amount of trastuzumab in association with an anthracycline to a patient in need thereof, in combination with an amount of dexrazoxane effective to ameliorate cardiotoxicity.

3. A method for treating a cancer, which comprises administering a therapeutically effective amount of trastuzumab in association with epirubicin to a patient in need thereof, in combination with an amount of dexrazoxane effective to ameliorate cardiotoxicity.

4. A method according to claim 1, wherein the cancer is a cancer overexpressing the human epidermal growth factor receptor 2 (HER2).

5. A method according to claim 4, wherein the cancer overexpressing the human epidermal growth factor receptor 2 (HER2) is selected from the group consisting of breast, uterine endometrium, pancreas, colon, ovaries, lung, stomach, salivary glands, head, and neck cancer.

6. A method according to claim 5, wherein the cancer is breast cancer.

7. A method according to claim 2, wherein the cancer is a cancer overexpressing the human epidermal growth factor receptor 2 (HER2).

8. A method according to claim 7, wherein the cancer overexpressing the human epidermal growth factor receptor 2 (HER2) is selected from the group consisting of breast, uterine endometrium, pancreas, colon, ovaries, lung, stomach, salivary glands, head, and neck cancer.

9. A method according to claim 8, wherein the cancer is breast cancer.

10. A method according to claim 3, wherein the cancer is a cancer overexpressing the human epidermal growth factor receptor 2 (HER2).

11. A method according to claim 10, wherein the cancer overexpressing the human epidermal growth factor receptor 2 (HER2) is breast, uterine endometrium, pancreas, colon, ovaries, lung, stomach, salivary glands, head, and neck cancer.

12. A method according to claim 11, wherein the cancer is breast cancer.

13. A method for ameliorating cardiotoxic effects caused by trastuzumab when administered alone, which comprises administering an effective amount of dexrazoxane.

14. A method for ameliorating cardiotoxic effects caused by trastuzumab when administered in combination with an anthracycline, which comprises administering an effective amount of dexrazoxane.

15. A method for ameliorating cardiotoxic effects caused by trastuzumab when administered in combination with epirubicin, which comprises administering an effective amount of dexrazoxane.

16. A kit comprising:
a. trastuzumab and a pharmaceutically acceptable carrier or diluent in a first unit dosage form;
b. dexrazoxane and a pharmaceutically acceptable carrier or diluent in a second unit dosage form; and
c. a container.

17. A kit comprising:
a. trastuzumab and a pharmaceutically acceptable carrier or diluent in a first unit dosage form;
b. an anthacycline and a pharmaceutically acceptable carrier or diluent in a second unit dosage form;
c. dexrazoxane and a pharmaceutically acceptable carrier or diluent in a third unit dosage form; and
d. a container.

18. A kit comprising:
a. trastuzumab and a pharmaceutically acceptable carrier or diluent in a first unit dosage form;
b. epirubicin and a pharmaceutically acceptable carrier or diluent in a second unit dosage form;
c. dexrazoxane and a pharmaceutically acceptable carrier or diluent in a third unit dosage form; and
d. a container.

19. A combined preparation for ameliorating cardiotoxicity selected from the group consisting of:
(a) trastuzumab and dexrzozane; and
(b) trastuzumab, anthracycline, and dexrazoxane,
wherein each component of the combined preparation is administered simultaneously, separately, or sequentially.

20. Dexrazoxane for use in the manufacture of a medicament for ameliorating cardiotoxicity induced by trastuzumab.

21. Dexrazoxane for use in the manufacture of a medicament for ameliorating cardiotoxicity induced by trastuzumab in conjunction with an anthracycline.

22. Dexrazoxane for use in the manufacture of a medicament for ameliorating cardiotoxicity induced by trastuzumab in conjunction with epirubicin.
